(19) United States
(12) Patent Application Publication
(10) Pub. No.: US 2008/0172019 A1
(43) Pub. Date: Jul. 17, 2008

(54) PAD

(76) Inventor: Yuan-Cheng Chien, Kaohsiung Hsien (TW)

Correspondence Address: PATENTTM.US, P. O. BOX 82788, PORTLAND, OR, 97282-0788, US

(21) Appl. No.: 11/622,428

(22) Filed: Jan. 11, 2007

(51) Int. Cl. A61F 13/15

(52) U.S. Cl. 604/385.04; 604/385.101; 604/385.01

(57) ABSTRACT

A pad has a rear end and an absorbing part. The absorbing part has an absorbing belt. The absorbing belt is mounted on the absorbing part and has a distal end. The distal end of the absorbing belt extends and protrudes from the rear end of the pad. Users can hold the distal end of the absorbing belt and adjust the absorbing belt to level at a crack between buttocks. Thus, menstrual blood flowing along the crack between buttocks will be absorbed by the absorbing belt and then flow along the absorbing belt to the absorbing part of the pad. Therefore, users do not worry that menstrual blood will flow out of the pad at night to dirt knickers and a bed sheet.

### BACKGROUND OF THE INVENTION

1. Field of Invention

The present invention relates to a pad, and more particularly to a pad that can prevent menstrual blood from flowing out of a rear of the pad.

2. Description of the Related Art

Ladies will worry about if pads are comfortable and can prevent menstrual blood from flowing out when ladies on their period. Thus, the pad has a front, a rear, two sides and an absorbing part. The rear of the pad may be elongated to prevent menstrual blood leaking out at night. Each side of the pad may have a wing to stick the pad securely on knickers, so ladies can exercise more freely. The absorbing part of the pad may have an all around flow guard to prevent menstrual blood from flowing out from the front, the rear and two sides of the pad. The pad may be scented to eliminate a peculiar smell.

However, an elongated pad for night can only prevent a part of menstrual blood from flowing out of the rear of the pad. Some menstrual blood will flow along a crack between buttocks and out of the pad when ladies sleep. Thus, menstrual blood will flow out of the pad to dirt knickers and a bed sheet and may remain in the crack between buttocks.

Someone invents a pad having a protrusion part formed on a central part of the absorbing part near the rear and protruding in the crack between buttocks to prevent the menstrual blood from flowing out of the pad and prevent menstrual blood from remaining in the crack of the buttocks. However, the protrusion part will be formed when the pad is unfolded, so the protrusion part is hard to be level at the crack between buttocks and is also hard to be adjusted. Moreover, when the user sleeps, she may turn aside and the menstrual blood may flow aside, so the protrusion part is useless. Thus, the pad with the protrusion part can not indeed prevent the menstrual blood from flowing out of the pad.

To overcome the shortcomings, the present invention provides a pad to mitigate or obviate the aforementioned.

### SUMMARY OF THE INVENTION

The primary objective of the present invention is to provide a pad that can prevent menstrual blood from flowing out of a rear of the pad.

To achieve the objective, the pad has a rear end and an absorbing part. The absorbing part has an absorbing belt. The absorbing belt is mounted on the absorbing part and has a distal end. The distal end of the absorbing belt extends and protrudes from the rear end of the pad. Users can hold the distal end of the absorbing belt and adjust the absorbing belt to level at a crack between buttocks. Thus, menstrual blood flowing along the crack between buttocks will be absorbed by the absorbing belt and then flow along the absorbing belt to the absorbing part of the pad. Therefore, users do not worry that menstrual blood will flow out of the pad at night to dirt knickers and a bed sheet.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a pad in accordance with the present invention with a sheet-shaped absorbing belt;

FIG. 2 is an operational perspective view of the pad in FIG. 1;

FIG. 3 is a perspective view of a pad in accordance with the present invention with a first configuration of a wave-shaped absorbing belt;

FIG. 4 is a perspective view of a pad in accordance with the present invention with a second configuration of a wave-shaped absorbing belt;

FIG. 5 is a perspective view of a pad in accordance with the present invention with a third configuration of a wave-shaped absorbing belt;

FIG. 6 is a perspective view of a pad in accordance with the present invention with a sheet-shaped absorbing belt having multiple equidistant protrusions;

FIG. 7 is a perspective view of a pad in accordance with the present invention with a sheet-shaped absorbing belt having multiple non-equidistant protrusions;

FIG. 8 is a perspective view of a pad in accordance with the present invention with a spiral-shaped absorbing belt;

FIG. 9 is a perspective view of a pad in accordance with the present invention with an absorbing belt being a foldable sheet;

FIG. 10 is a cross sectional end view of the pad in FIG. 9;

FIG. 11 is a perspective view of a pad in accordance with the present invention with an elongated absorbing belt; and

FIG. 12 is an operational perspective view of the pad in FIG. 11.

### DETAILED DESCRIPTION OF THE INVENTION

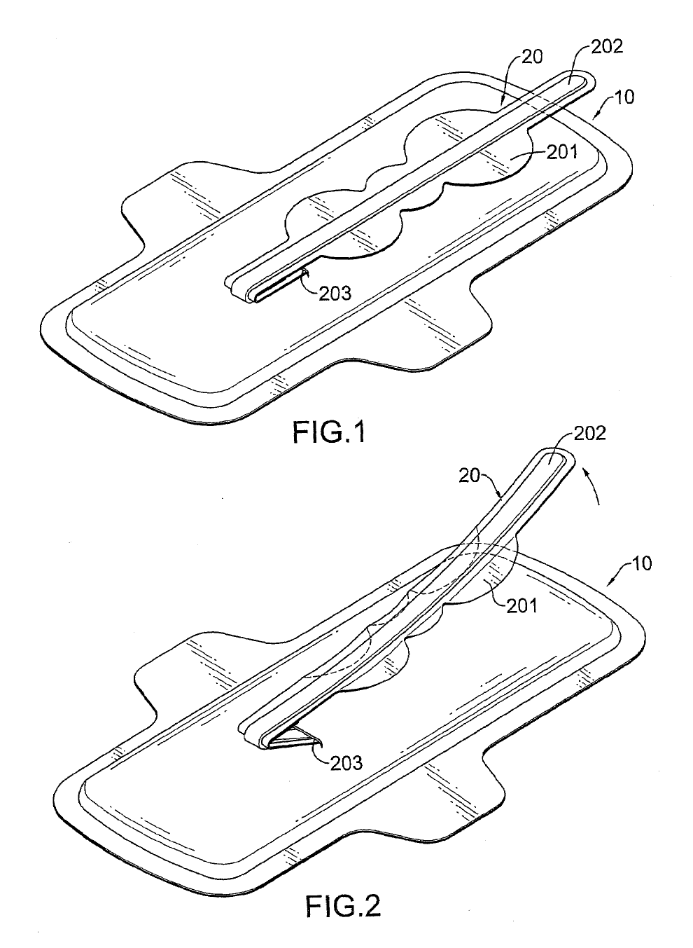

With reference to FIG. 1, a pad (10) in accordance with the present invention has a bottom, a front end, a rear end, two sides and an absorbing part.

The bottom contacts with a user's knickers and has adhesives to allow the pad (10) to stick securely on the knickers.

The rear end of the pad may be broadened or elongated.

Each side may have a wing. The wing has a lower surface. The lower surface of the wing has adhesives to stick on the knickers to allow the pad to stick on the knickers securely.

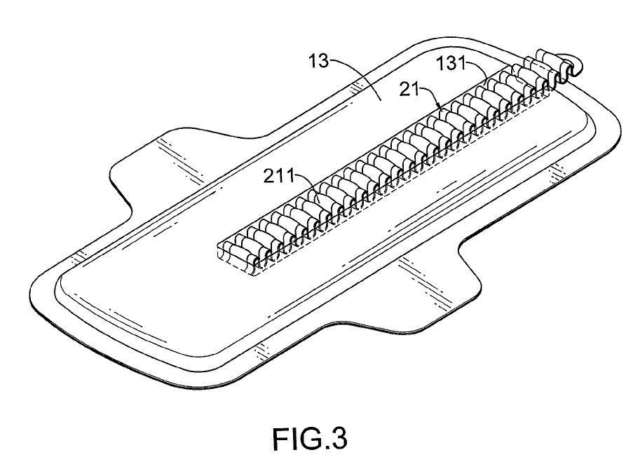

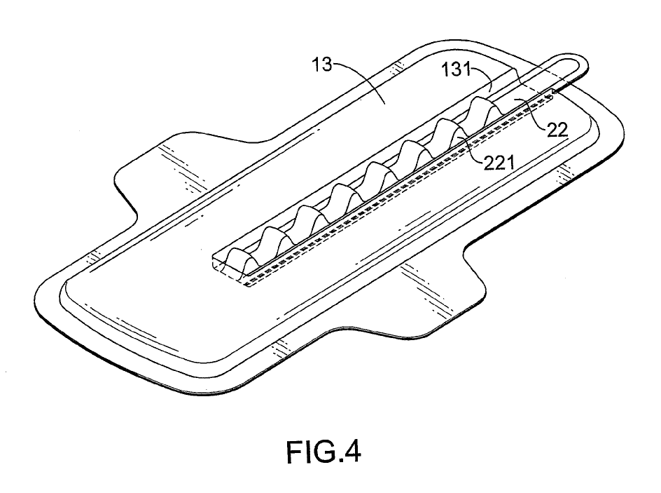

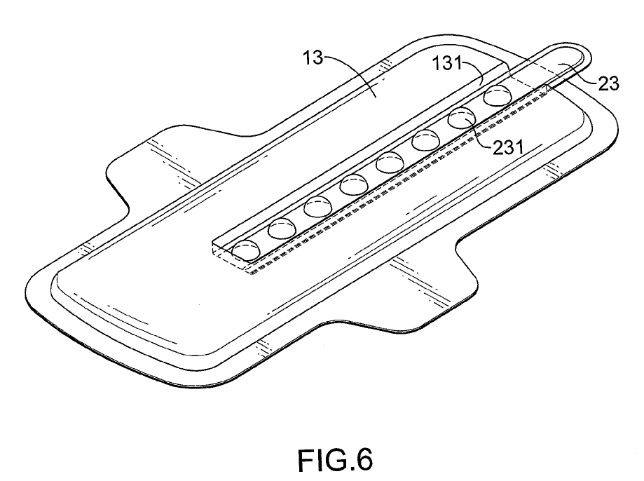

With further reference to FIGS. 3, 4, 6, 8 and 9, the absorbing part has an inner space, an upper surface, a groove (131) and an absorbing belt (20-26).

The inner space has multiple absorbing layers to absorb menstrual blood.

The upper surface is made of a nonwoven material to make the user comfortable.

The groove (131) is formed in the absorbing part.

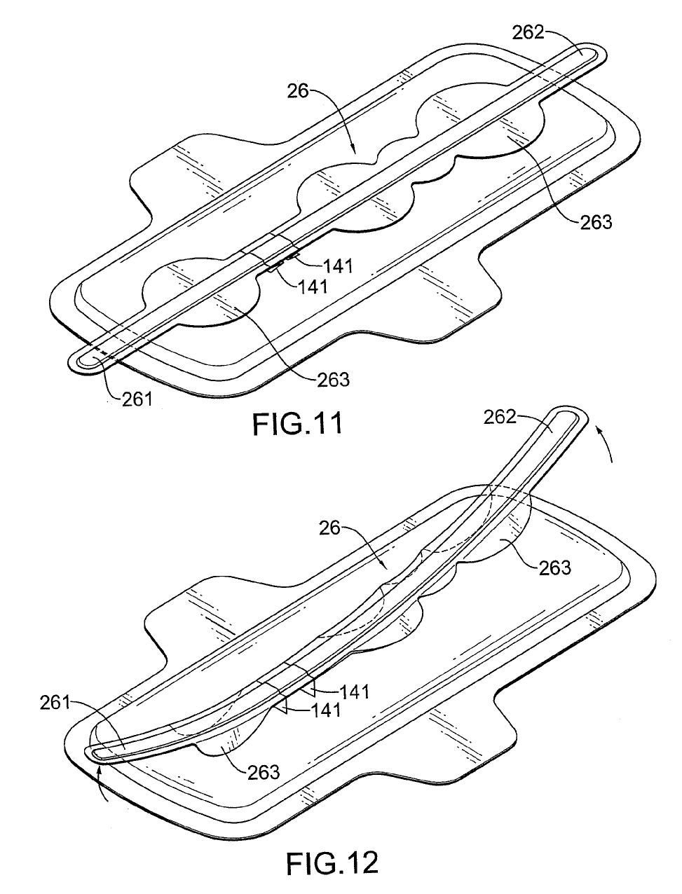

With further reference to FIGS. 2, 11 and 12, the absorbing belt (20-26) is mounted on the upper surface of the absorbing part and may be mounted in the groove (131). The absorbing belt (20-26) has a proximal end (203, 261), a distal end (202, 262) and at least one connector (141). The proximal end (203, 261) of the absorbing belt (20-26) may be mounted on the upper surface of the absorbing part directly and may be elongated to extend and protrude from the front end to allow a user to hold and pull up the absorbing belt (25, 26) to level at a crack between buttocks. The distal end (202, 262) extends and protrudes from the rear end of the pad (10) to allow a user to hold and pull up the absorbing belt (20-26) to level at a crack between buttocks, so the absorbing belt (20-26) can be mounted in the crack between buttocks to absorb menstrual blood and guide menstrual blood flow along the absorbing belt (20-26) to the absorbing part. The connector (141) may be mounted on the upper surface of the absorbing part to connect the absorbing belt (20-26) with the upper surface of the absorbing part and may be mounted on the groove (131) to connect the absorbing belt (20-26) with the absorbing part.

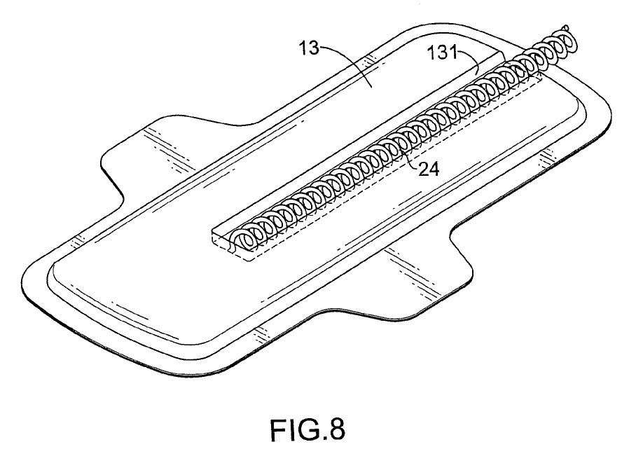

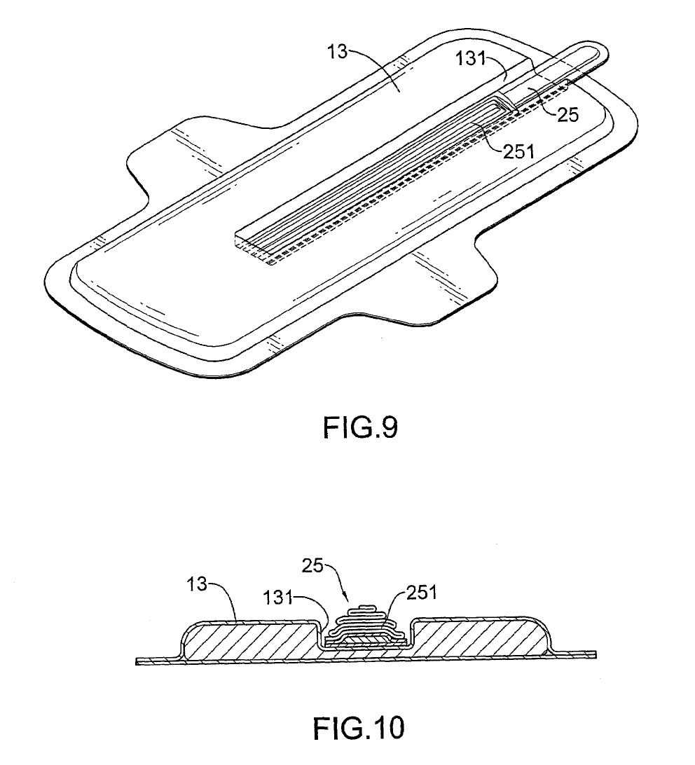

The absorbing belt (20-26) may be a sheet-shaped absorbing belt (20, 23, 26) in FIGS. 1, 6 and 11, a wave-shaped absorbing belt (21, 22) in FIGS. 3 to 5, a spiral-shaped absorbing belt (24) in FIG. 8, may have a foldable absorbing layer (251) in FIG. 9.

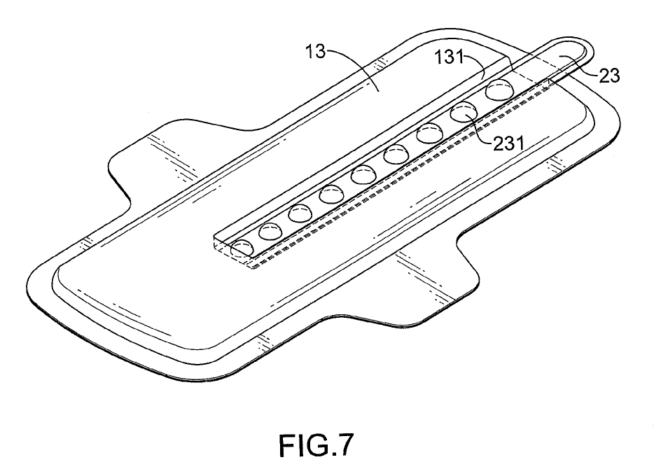

With further reference to FIG. 7, the sheet-shaped absorbing belt (20, 23, 26) has two sides and multiple absorbing protrusions (231). Each side has at least one absorbing tab (201, 263) to absorb menstrual blood more. The absorbing protrusions (231) are formed on and aligned on the sheet absorbing belt (20, 23) to absorb menstrual blood more. A dimension of the protrusions may be equal or become larger from the proximal end (203, 261) to the distal end (202, 262).

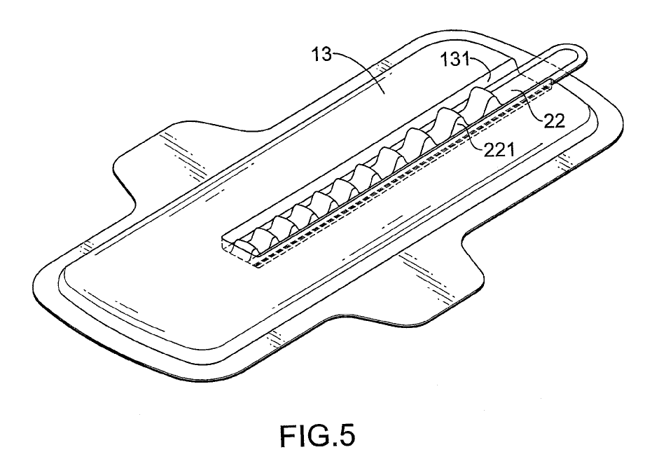

With further reference to FIG. 5, the wave-shaped absorbing belt (21, 22) has multiple wave bands (211, 221). A wavelength between two adjacent wave bands (211, 221) may be equal or become longer geometrically from the proximal end (203, 261) to the distal end (202, 262).

With further reference to FIG. 10, the absorbing belt (25) has the foldable absorbing layer (251). The foldable absorbing layer (251) is folded in the groove (131) before the pad (10) is used and has a top, a bottom and an end cross-section. The bottom is wider than the top. The end cross-section is trapezoid-shaped.

Users can hold distal end (202, 262) of the absorbing belt (20-26) and adjust the absorbing belt (20-26) to level at the crack between buttocks, so menstrual blood flowing along the crack between buttocks will be absorbed by the absorbing belt (20-26) and then flow along the absorbing belt (20-26) to the absorbing part of the pad (10). Therefore, users will not worry that menstrual blood will flow out of the pad (10) at night to dirt knickers and a bed sheet.

Even though numerous characteristics and advantages of the present invention have been set forth in the foregoing description, together with details of the structure and function of the invention, the disclosure is illustrative only. Changes may be made in detail, especially in matters of shape, size and arrangement of parts within the principles of the invention to the full extent indicated by the broad general meaning of the terms in which the appended claims are expressed.

1. A pad comprising:
a rear end;
an absorbing part having
an upper surface; and
an absorbing belt being connected to the upper surface of the absorbing part and having
a proximal end being connected directly to the upper surface of the absorbing part; and
a distal end extending and protruding out of the rear end of the pad, wherein the absorbing belt is connected to the absorbing part only at the proximal end.

2. (canceled)

3. The pad as claimed in claim 1 further having a front end, wherein
the absorbing belt further has at least one connector being only connected the proximal end of the absorbing belt and the absorbing part of the pad; and
the proximal end having a protruding part that is connected to the proximal end, is elongated to extend and protrude out of the front end

4. (canceled)

5. The pad as claimed in claim 1, wherein the absorbing part further has a groove formed in the absorbing part and the absorbing belt is mounted in the groove.

6. The pad as claimed in claim 1, wherein the absorbing belt is a sheet and has two sides and each side has at least one absorbing tab.

7. The pad as claimed in claim 1, wherein the absorbing belt is a sheet and has multiple absorbing protrusions formed on and aligned on the sheet.

8. The pad as claimed in claim 7, wherein every two protrusions have a distance between the protrusions being equal.

9. The pad as claimed in claim 7, wherein every two protrusions have a dimension of the protrusions being larger from the proximal end to the distal end.

10. The pad as claimed in claim 1, wherein the absorbing belt is wave-shaped.

11. The pad as claimed in claim 10, wherein the absorbing belt has multiple wave bands and every two adjacent wave bands has a wavelength between the two adjacent wave bands being equal.

12. The pad as claimed in claim 10, wherein the absorbing belt has multiple wave bands and every two adjacent wave bands has a wavelength between the two adjacent wave bands being longer geometrically from the proximal end to the distal end.

13. The pad as claimed in claim 1, wherein the absorbing belt is spiral-shaped.

14. The pad as claimed in claim 5, wherein the absorbing belt has a foldable absorbing layer being folded in the groove before the pad is used.

15. The pad as claimed in claim 14, wherein the absorbing belt has
a top;
a bottom being wider than the top; and
an end cross-section being trapezoid-shaped.

16. The pad as claimed in claim 1, wherein the upper surface of the absorbing part is made of a nonwoven material.

17. The pad as claimed in claim 1 further having two sides and each side having a wing.
